(19) United States
(12) Patent Application Publication
(10) Pub. No.: US 2007/0010815 A1
(43) Pub. Date: Jan. 11, 2007

(54) Fixation systems with modulated stiffness

(75) Inventor: Fred Molz, Collierville, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/170,554

(22) Filed: Jun. 30, 2005

(51) Int. Cl. A61F 2/30

(52) U.S. Cl. 606/69

(57) ABSTRACT

The invention provides a fixation device for implantation in a patient to provide support to a body component. The fixation device includes an outer construct forming a pocket and a slurry disposed in the pocket. The slurry is comprised of magnetic particles. The slurry is responsive to an electric field to modulate stiffness of the slurry. A system is provided to generate the electric field. Further, a method is provided to use the fixation device.

### FIELD OF THE INVENTION

Embodiments of the invention relate to fixation systems. More particularly, the invention relates to fusion and fracture fixation devices in which the stiffness of the fixation device is modulated.

### BACKGROUND

Damage to one or more components of the body, such as the spinal column, may result from disease or trauma. To prevent further damage and overcome some of the symptoms resulting from a damaged body component, a fixation device may be installed to stabilize the body component.

In illustrative reference to the spinal column, the spinal (vertebral) column is a biomechanical structure composed primarily of ligaments, muscles, vertebrae, and intervertebral discs. The biomechanical functions of the spinal column include (i) support of the body; (ii) regulation of motion between the head, trunk, arms, pelvis, and legs; and (iii) protection of the spinal cord and the nerve roots.

A spinal fixation device generally includes stabilizing elements, such as rods or plates, attached by anchors to the vertebrae in the section of the vertebral column that is to be stabilized. The spinal fixation device restricts the movement of the fixed vertebrae relative to one another and supports at least a part of the stresses that would otherwise be imparted to the vertebral column. Typically, the stabilizing element is rigid and inflexible. However, there are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In response, flexible spinal fixation devices have been employed. These devices are designed to support at least a portion of the stresses imparted to the vertebral column but also allow a degree of movement. In this way, flexible spinal fixation devices avoid some of the disadvantages of rigid spinal fixation devices. However, there are problems with flexible spinal fixation devices. Flexible spinal fixation devices may also be too rigid, or alternatively too flexible. Damage to body components vary widely. There is a need for a fixation device that can correspondingly vary. In particular, there is need for a fixation device having stiffness that can be controlled.

The description herein of problems and disadvantages of known apparatuses, methods, and devices is not intended to limit the invention to the exclusion of these known entities. Indeed, embodiments of the invention may include, -or be used in conjunction with, one or more of known apparatus, methods, and devices without suffering from the disadvantages and problems noted herein.

### SUMMARY OF THE INVENTION

The invention provides a fixation device for implantation in a patient to provide support to a body component. The fixation device includes an outer construct forming a pocket and a slurry disposed in the pocket. The slurry may be comprised of magnetic particles. The slurry is responsive to an electric field to modulate stiffness of the slurry, and in turn the fixation device. A system is provided to generate the electric field. Further, a method is provided to use the fixation device.

### BRIEF DESCRIPTION OF THE DRAWINGS

The present invention can be more fully understood by reading the following detailed description together with the accompanying drawings, in which like reference indicators are used to designate like elements, and in which:

FIG. 1 is schematic diagram of an external fixation system with fixation device in accordance with one embodiment of the invention;

FIG. 2(a) is a diagram showing the fixation device of FIG. 1 in further detail in accordance with one embodiment of the invention;

FIG. 2(b) is a cross section of the fixation device of FIG. 2(a) along line 2(b)-2(b);

FIG. 3 is schematic diagram of an internal fixation system with fixation device in accordance with one embodiment of the invention;

FIG. 4 is a block diagram of the implanted electric field generation portion of FIG. 3 shown in further detail in accordance with one embodiment of the invention;

FIG. 5 is a flowchart illustrating a process for using a fixation device with an electric field applied externally, in accordance with one embodiment of the invention;

FIG. 6 is a flowchart illustrating a process for using a fixation device with an electric field applied internally, in accordance with one embodiment of the invention;

FIG. 7 is a schematic diagram of a fixation system, including a fixation device, with the electric field turned off in accordance with one embodiment of the invention;

FIG. 8 is a schematic diagram of the fixation system of FIG. 7 with the electric field turned on in accordance with one embodiment of the invention;

FIG. 9 is a schematic diagram of a fixation system, including a fixation device having two different types of magnetic material in respective pockets, in accordance with one embodiment of the invention; and

FIG. 10 is a schematic diagram of a fixation system having two different electric fields in accordance with one embodiment of the invention;

FIG. 11 is a further schematic diagram of a fixation system having two different electric fields in accordance with one embodiment of the invention; and

FIG. 12 is a schematic diagram showing a further fixation device in accordance with one embodiment of the invention.

### DETAILED DESCRIPTION OF THE EMBODIMENTS

Hereinafter, aspects of fixation devices in accordance with various embodiments of the invention will be described. As used herein, any term in the singular may be interpreted to be in the plural, and alternatively, any term in the plural may be interpreted to be in the singular.

The following description is intended to convey a thorough understanding of the various embodiments of the invention by providing a number of specific embodiments and details involving fixation devices with modulated stiffness. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

It is a feature of an embodiment of the present invention to provide a flexible fixation device that is filled with a slurry of magnetic or conductive particles. An electric field is applied to the fixation device, during use of the device, to control or modulate the stiffness of the device. An example would be a spinal rod for fusion. After implantation, an electric field is applied to make the rod stiff while fusion of the bone, for example, occurs. After or during fusion of the bone or other body component, the field is slowly removed, providing increased loading of the fusion mass of the bone, i.e., calcification starts. This may involve a period of weeks or months. The result is a healthy fusion mass without stress shielding caused by remaining steel hardware. Accordingly, as the bone, for example, heals—the field may be slowly removed, i.e., the strength of the field may be decreased or decayed using a suitable controller, e.g., a potentiometer.

In embodiments, the electric field can be supplied by a variety of arrangements, including an external corset or an implanted device with a controller and rechargeable batteries, for example. One example is a flexible spinal construct that is implanted to stabilize the spine, provide mobility, and relieve pain. The stiffness of the construct is set at a level that relieves pain while providing as much motion as possible. Thus, the stiffness of the construct may be set to provide a period of healing followed by a return to motion that is easily tolerated by the patient and is pain free.

The invention provides a flexible device that can be customized to treat individual patients. The technology may be applied to a variety of support devices such as nucleus replacements, intervertebral disc designs, vertebralplasty, and general orthopaedic devices, for example.

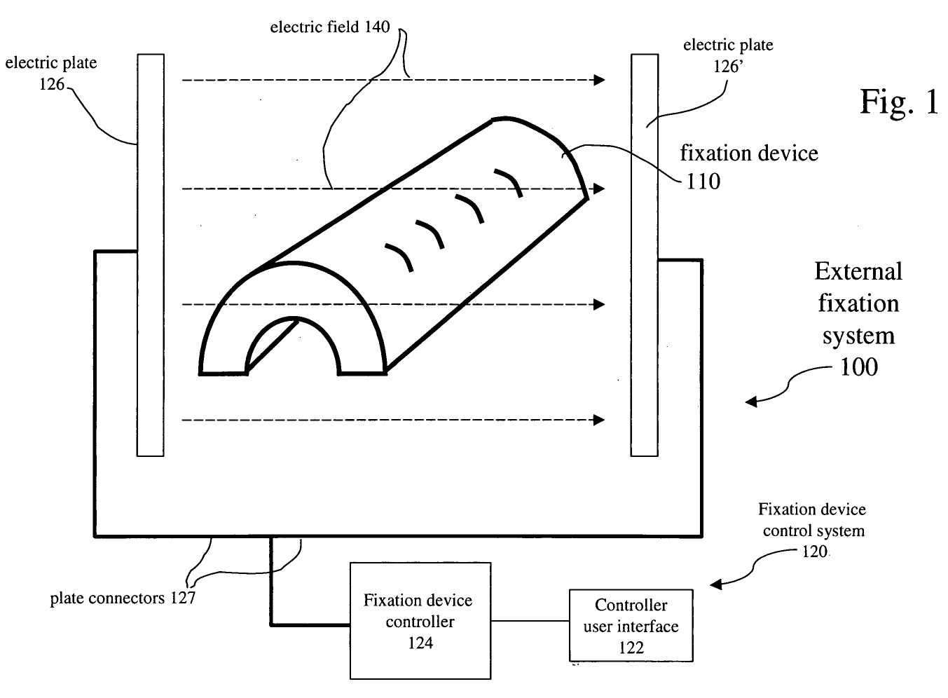

In further description of embodiments, FIG. 1 is schematic diagram of an external fixation system 100 in accordance with one embodiment of the invention. The external fixation system 100 includes a fixation device 110 and a fixation device control system 120. In this example, the magnetic field is applied to the fixation device externally of the patient in which the fixation device is disposed.

The fixation device control system 120 includes a fixation device controller 124 and electric plates (126, 126′). The fixation device controller 124 controls the application of an electric field 140 through the fixation device 110. The application of the electric field 140 is applied to a slurry in the fixation device 110. In embodiments, the slurry make-up can vary from a ferromagnetic powder or particles encased in a flexible bag to a solution of ferromagnetic powder and a liquid encased in a polymeric bag, for example. Further, it is contemplated that ferromagnetic liquids may be utilized as a slurry in one embodiment. However, other materials reacting to the electric field in the same manner as ferromagnetic material may also be used.

The electric plates (126, 126′) are shown in FIG. 1 as a pair of plates opposed to each other. However, a variety of arrangements of electric plates (126) might be used, as is desired, or some other arrangement to generate the electric field. Thus, the shape or number of plates might be varied, for example. In the embodiment of FIG. 1, the fixation device controller 124 is connected to the electric plates (126, 126′) via plate connectors 127.

A user, e.g. a human operator, interfaces with the fixation device controller 124 using a controller user interface 122. For example, the controller user interface 122 might be a computer, with keyboard and mouse, which is operatively connected to the fixation device controller 124.

In operation, the user controls, e.g. programs, the fixation device controller 124 to apply the electric field 140 from one electric plate 126 to an opposing electric plate 126′. The fixation device 110 is disposed in the path of the electric field 140. As described herein, the fixation device 110 is filled with a slurry of magnetic particles. In the time that the slurry is exposed to the electric field, the rigidity of the slurry is increased, i.e., based on the strength of the magnetic field. As a result, the rigidity of the fixation device 110 is modulated. The exposure of the slurry to the electric field 140 may be modulated by either varying the intensity of the electric field and/or varying the duration of time that the fixation device 110 is exposed in the electric field, for example.

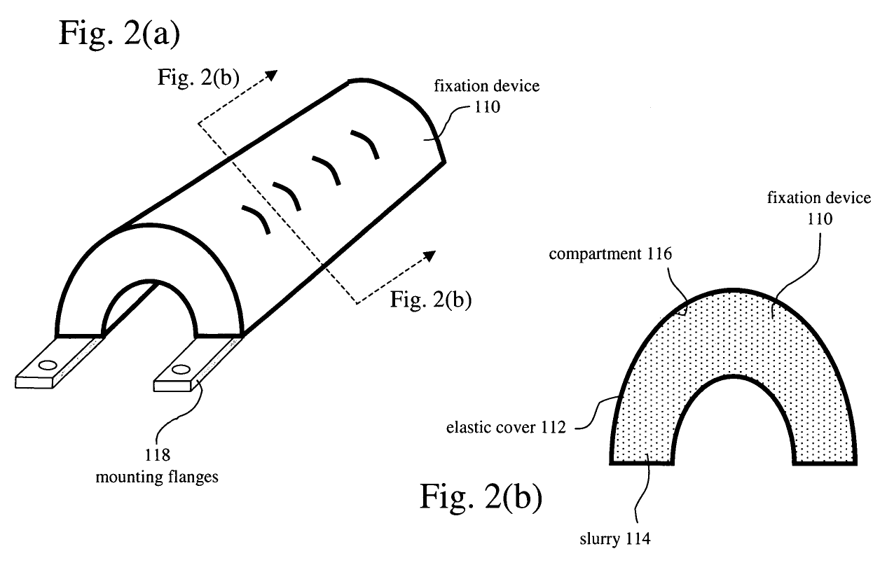

FIG. 2(a) is a diagram showing the fixation device 110 of FIG. 1 in further detail in accordance with one embodiment. FIG. 2(b) is a cross section of the fixation device of FIG. 2(a) along line 2(b)-2(b). As shown, the fixation device 110 includes a compartment or pocket 116 formed within an elastic cover 112. The compartment 116 is filled with a slurry of magnetic particles 114. The slurry of magnetic particles 114 is disposed in and contained by the compartment 116.

The elastic cover 112 may be constructed of any suitable material, such as plastic, polymer and/or rubber, for example. However, the elastic cover 112 should typically be constructed of material having some elasticity. That is, the rigidness of at least a part of the fixation device 110 should be dictated by the properties of the slurry (and the magnetic field applied to the slurry) and not the rigidity of the elastic cover 112.

In accordance with one embodiment, only a portion of a particular fixation device 110 contains the slurry of magnetic particles 114. Other portions of a particular fixation device 110 may be constructed so as to provide materials of a different rigidity. Alternatively, other portions of a fixation device 110 may be provided for connection to either a body component, e.g. a bone and/or to another fixation device. Illustratively, FIG. 2(a) shows the fixation device 110 may be provided with mounting flanges 118. The mounting flanges 118 may provide mounting of the fixation device 110 to either a body component and/or another fixation device, for example.

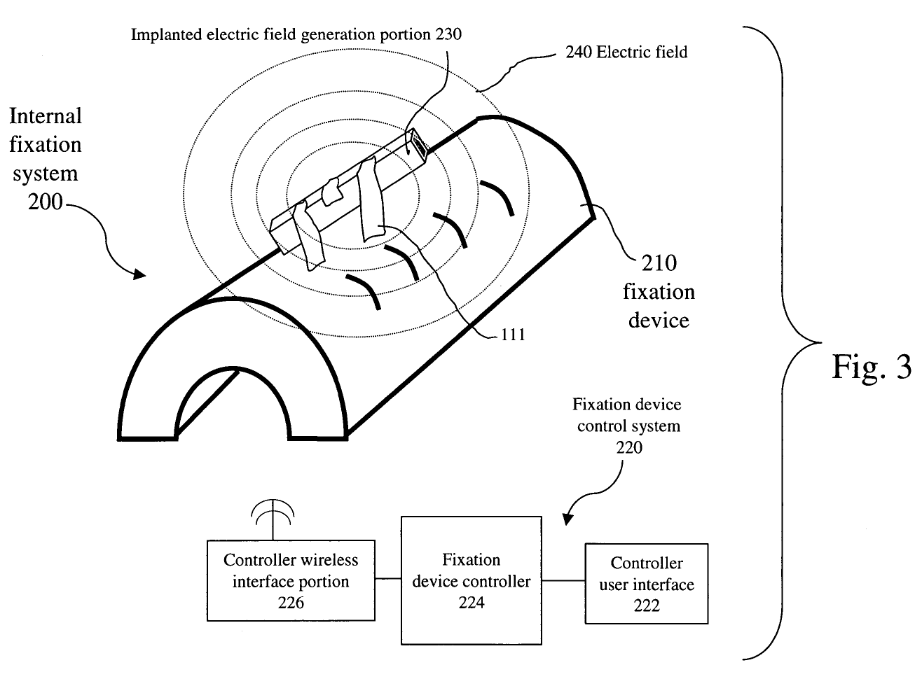

FIG. 3 is schematic diagram of an internal fixation system 200 in accordance with one embodiment of the invention. The internal fixation system 200 includes the fixation device 210 and a fixation device control system 220. The structure of the fixation device 110 may be the same as that discussed above or of different construction.

The internal fixation system 200, and specifically the fixation device control system 220, includes a controller user interface 222, a fixation device controller 224 and a controller wireless interface portion 226. The controller user interface 222 provides a user interface by which a user may operate the fixation device controller 224, i.e., control or program the fixation device controller 224 to control the application of an electric field 240.

The internal fixation system 200 further includes an implanted electric field generation portion 230, which is positioned adjacent the fixation device 210, as desired. For example, the implanted electric field generation portion 230 may be connected to the fixation device 210 via suitable tape 111 or in some other manner.

In this embodiment, the fixation device controller 224 communicates with the implanted electric field generation portion 230 wirelessly by using the controller wireless interface portion 226. Suitable known wireless technology may be utilized.

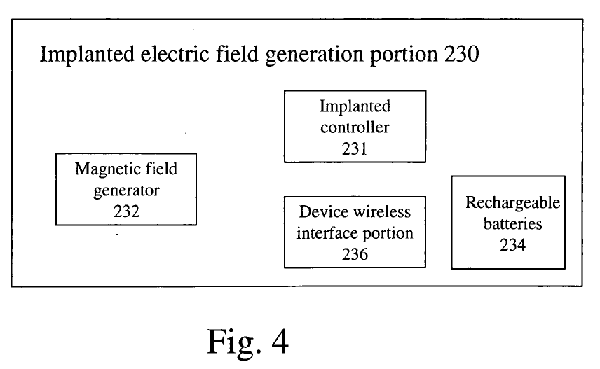

FIG. 4 is a block diagram of the implanted electric field generation portion 230 shown in further detail in accordance with one embodiment.

The implanted electric field generation portion 230 includes an implanted controller 231 to control operation of the implanted electric field generation portion 230. The implanted controller 231 communicates with the fixation device controller 224 using a device wireless interface portion 236 and the controller wireless interface portion 226. The implanted electric field generation portion 230 includes rechargeable batteries 234 as a power source.

The implanted electric field generation portion 230 also includes an electric field generator 232. The electric field generator 232 generates an electric field 240 as desired, as shown in FIG. 3.

In the embodiment of FIG. 3, the fixation device controller 224 wirelessly communicates with the implanted electric field generation portion 230 so as to control the generation of the electric field 240. However, in another embodiment, it is appreciated that the implanted electric field generation portion 230 may simply be programmed to generate a desired electric field so as to fuse the slurry 114, i.e., apply an electric field across the slurry so as to make the slurry rigid. In such embodiment, the implanted electric field generation portion 230 would be placed within the patient (adjacent-the fixation device 210) and thereafter would generate the electric field 240 independently of any external control.

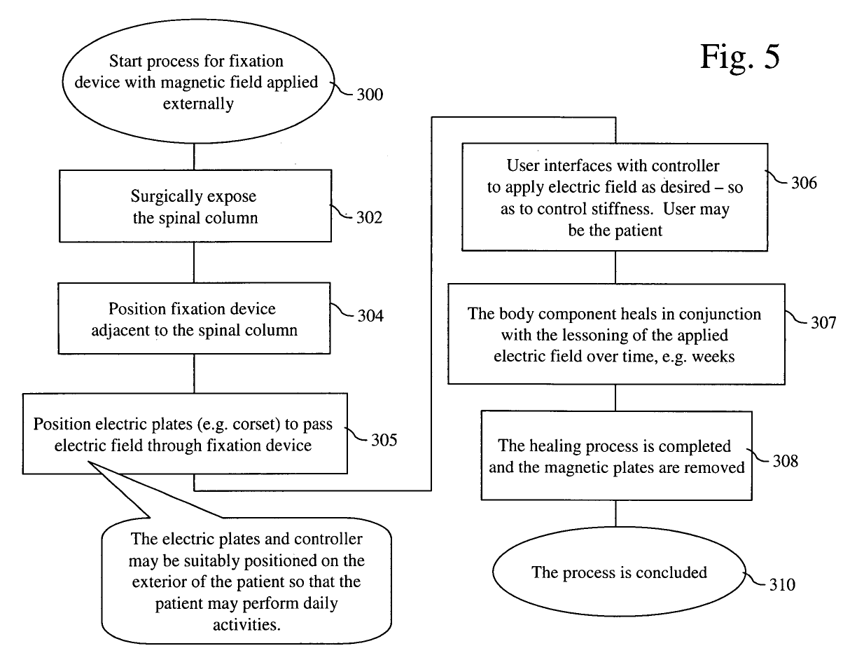

FIG. 5 is a flowchart illustrating a process for using a fixation device with an electric field applied externally. In the example of FIG. 5, the fixation device is-applied to a spinal column and the external fixation system 100 of FIG. 1 is utilized. As shown in FIG. 5, the process starts in step 300 and passes to step 302. In step 302, the patient is surgically prepared for insertion of the fixation device 11 0, i.e., in this example, the spinal column is exposed. Then, in step 304, the fixation device is positioned adjacent to the spinal column. After step 304, the process passes to step 305.

In step 305, the electric plates 126 are positioned to pass an electric field through the fixation device 110. The plates may be positioned so as to allow the patient to perform normal activities. Then, the external fixation system 100 is ready to apply the electric field to the fixation device 110. In step 306, the user interfaces with the fixation device controller 124 to apply the electric field to the fixation device 110, which contains the slurry of magnetic particles. The electric field is applied in a manner to control the intensity of the electric field or the duration of the electric field. In this manner the stiffness of the fixation device 1 10 is controlled by exposing the slurry to the electric field.

In step 307, the body component heals in conjunction with the lessoning of the applied electric field over time, e.g. weeks or months. That is, as the body component heals, the rigidity if the fixation device is lessoned, thus transferring more load onto the particular body component. Then, in step 308, the healing process is deemed to be competed. Accordingly, the magnetic plates are removed. Then, the process is concluded in step 310. The fixation device may also be removed from the patient at some appropriate time, as desired.

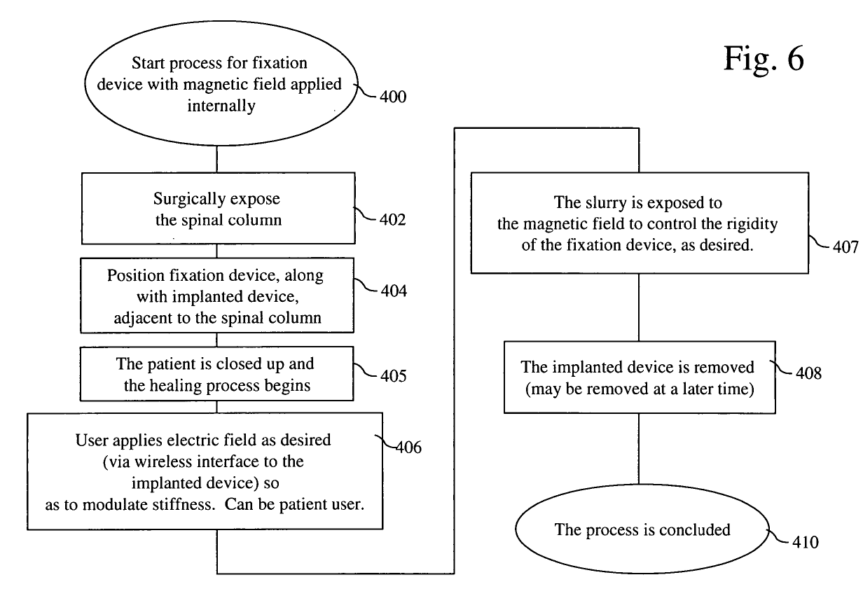

FIG. 6 is a flowchart illustrating a process for using a fixation device with an electric field applied internally, in accordance with one embodiment. In this example, the internal fixation system 200 of FIG. 3 is utilized.

The process of FIG. 6 starts in step 400 and passes to step 402. In step 402, the patient is surgically prepared to receive the fixation device 110, i.e., the spinal column is exposed, fore example. Then, in step 404, the fixation device 1 10 is positioned in the desired position in the patient, e.g., adjacent the spinal column. Further, the implanted electric field generation portion 230 is positioned adjacent the fixation device 110. For example, the implanted electric field generation portion 230 may be connected to the fixation device 110 via suitable tape 111, as noted above. Then, in step 405, the patient is closed up and the healing process begins.

In step 406, the user applies an electric field 231 as desired by interfacing with the controller user interface 222. That is, the user interfaces with the fixation device controller 224 using the controller user interface 222. In turn, the fixation device controller 224 communicates with the implanted electric field generation portion 230 via wireless interface, i.e., so as to remotely control operation of the implanted electric field generation portion 230.

In step 407 of FIG. 6, the slurry is exposed to the electric field so as to control the rigidity of the fixation device, as desired. Then, in step 408, the implanted electric field generation portion 230 with fixation device is removed, i.e., once healing of the patient is completed, which may be weeks or months. After step 408, the process passes to step 410. In step 410, the process is concluded.

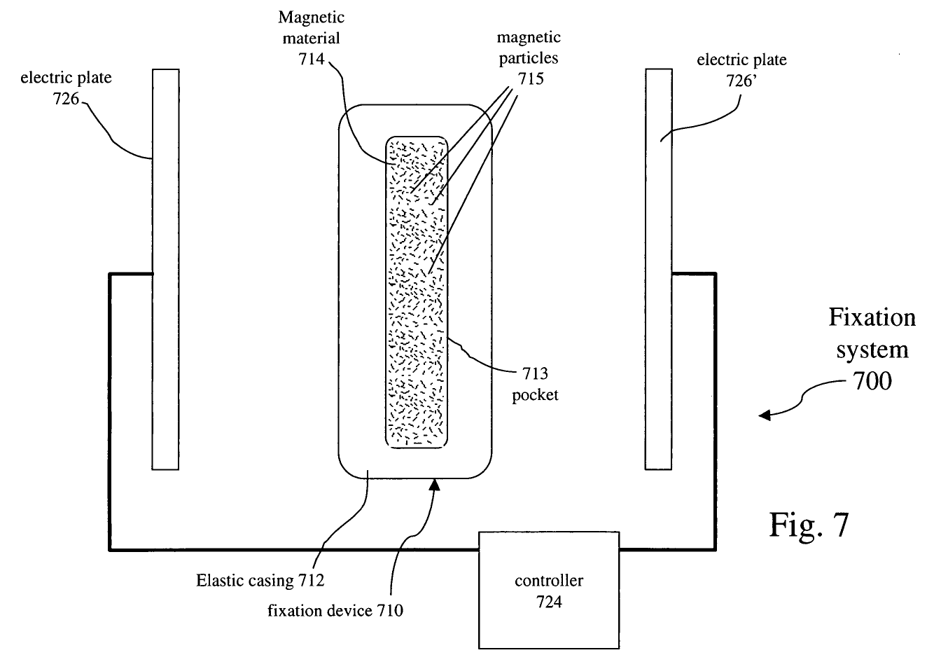

FIG. 7 is a schematic diagram of a fixation system, including a fixation device, with the electric field turned off in accordance with one embodiment of the invention. In further detail, the fixation system 700 includes a controller 724. The controller 724 controls the generation of an electric field from a first electric plate 726 to a second electric plate 726′. The fixation system 700 further includes a fixation device 710. The fixation device 710 includes an elastic casing 712. A pocket 713 is disposed in the elastic casing 712. Magnetic material 714 (containing magnetic particles 715) is contained in the pocket 713.

The fixation device 710 is disposed between the electric plates (726, 726′) such that once an electric field is applied, the electric field will pass through the magnetic material 714 disposed in the fixation device 710. The magnetic material 714 may be in the form of a suitable slurry of ferro-magnetic material. For example, the magnetic material 714 might be comprised of magnetic particles 715 in a suitable solution.

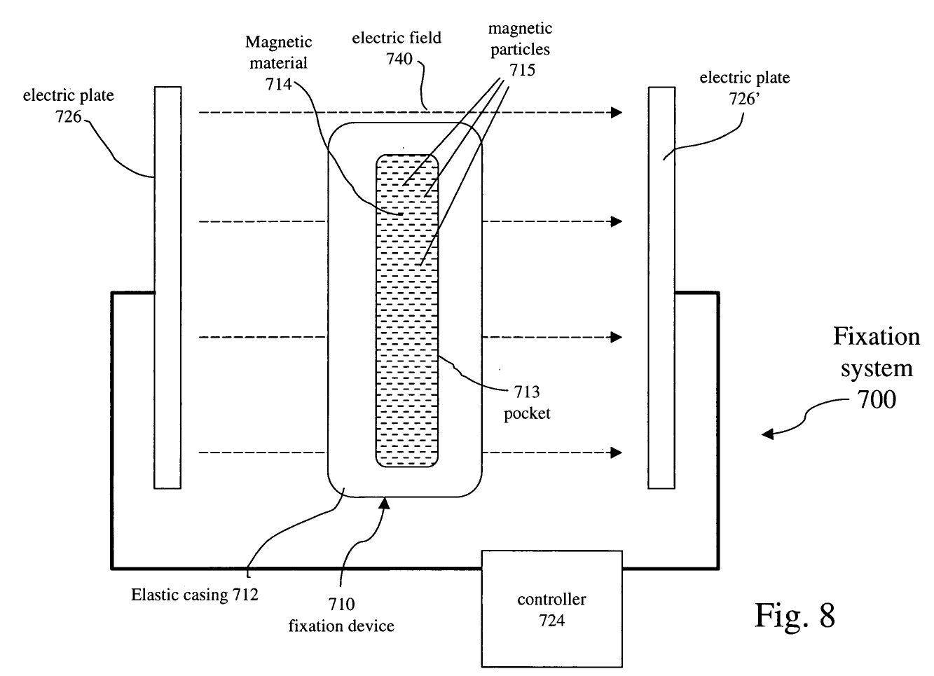

FIG. 8 is a schematic diagram of the fixation system of FIG. 7. However, in FIG. 8 the electric field 740 between the electric plate 726 and the electric plate 726′ is turned on. Accordingly, the electric field 740 causes the magnetic material 714, and specifically the particles 715 in the magnetic material 714, to be oriented in a direction aligned with the electric field 740. With a given magnetic material 714, a stronger electric field 740 will result in the magnetic particles 715 being more resistance to a change in orientation out of line with the magnetic material 714. Accordingly, as the magnetic particles 715 are more resistant to change in orientation out of line with the electric field 740, the fixation device 710 will be more resistance to deformation, i.e., to a change in shape. A fixation device 710 that is more resistant to a change in shape will in turn provide more support to a particular body component being supported. Thus, the support to a particular body component that is provided by the fixation device 710 may be varied by varying the electric field 740. In short, the stronger the electric field 740, the stronger the support provided.

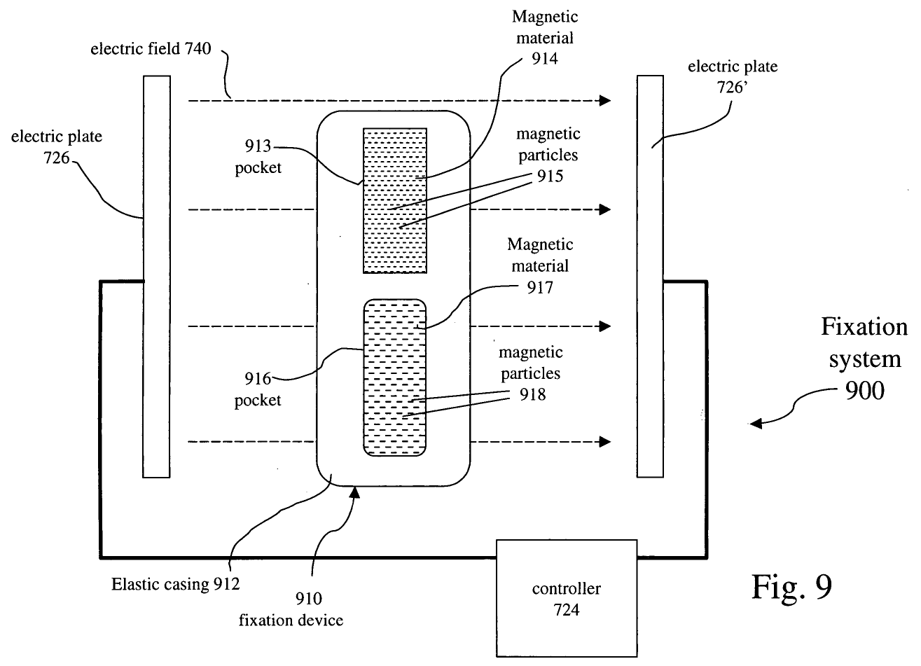

FIG. 9 is a schematic diagram of a fixation system 900 in accordance with one embodiment of the invention. The controller 724 and the electric plates (726, 726′) of the fixation system 900 may be the same as that shown in FIGS. 7 and 8. The fixation system 900 of FIG. 9 is provided with a fixation device 910. The fixation device 910 includes two pockets (913, 916) disposed in an elastic casing 912. The elastic casing 912 may be attached to other support components and/or to body components in any suitable manner, e.g., such as using known fastening devices.

In the elastic casing 912, the pocket 913 contains a magnetic material 914. The magnetic material 914 is comprised of magnetic particles 915 that are supported in a suitable solution, i.e., so as to form a slurry mixture.

The elastic casing 912, also includes the pocket 916. The pocket 916 contains a magnetic material 917. The magnetic material 917 is comprised of magnetic particles 918 that are supported in a suitable solution, i.e., so as to also form a slurry mixture.

As depicted in the drawings, the magnetic particles 915 are different than the magnetic particles 918. For example, as compared with the magnetic particles 918, the magnetic particles 915 might be a different material, a different size, or vary in some other manner. As a result, the manner in which the magnetic particles 915 react to the electric field 740 will be different than the manner in which the magnetic particles 918 react, i.e., and in particular the respective rigidity provided by the magnetic particles (915, 918) will be different. For example, the magnetic particles 918 are depicted in FIG. 9 as having a larger size and may therefore provide more rigidity. However, it is appreciated that a wide variety of properties may effect the particular rigidity provided by a slurry, i.e., such as the composition, size, and shape of the particular magnetic particles 918 and the solution in which such magnetic particles are disposed.

Accordingly, in this example, the magnetic particles 918 provide more rigidity to the fixation device 910 than do the magnetic particles 915 while the electric field 740 is being applied. As a result, the portion of the elastic casing 912 surrounding the pocket 916 will be more rigid as compared with the portion of the fixation device 910 surrounding the pocket 913. It is of course appreciated that various attributes of the fixation device 910 may be varied as desired, including the number of pockets, the composition of the magnetic particles and slurry in the pockets, the number of pockets that have different slurry, the shape of the pockets and the relative orientation of the pockets, for example.

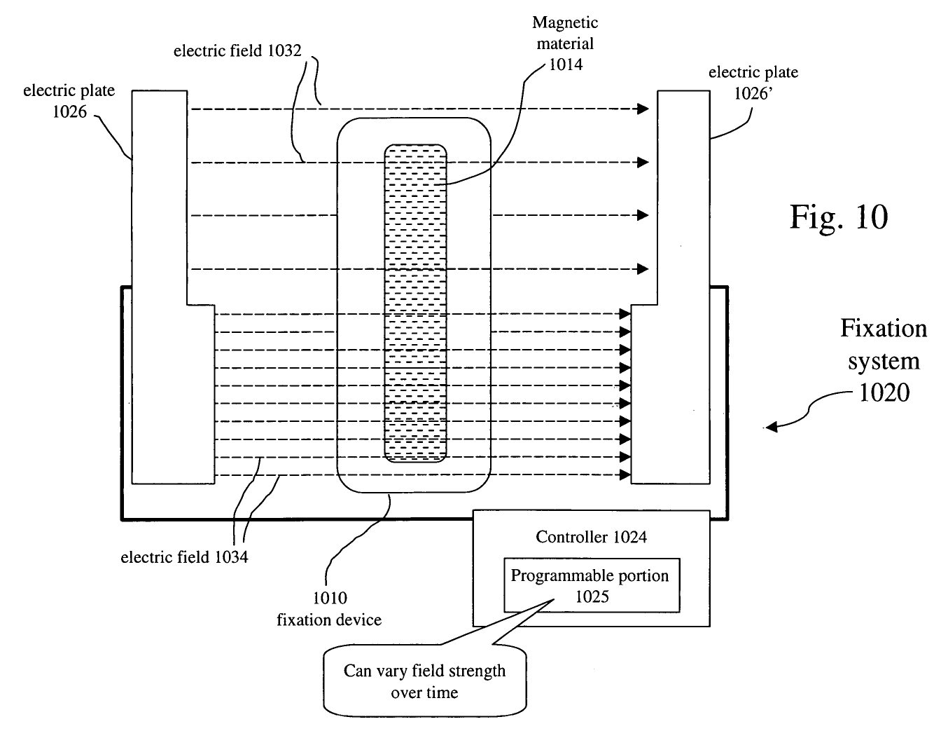

FIG. 10 is a schematic diagram of a fixation system 1020 having two different electric fields in accordance with one embodiment of the invention. As shown in FIG. 10, the fixation system 1020 includes a controller 1024. The controller 1024 controls the operation of two electric plates (1026, 1026′) that generate two electric fields (1032, 1034). As depicted in FIG. 10, the electric fields (1032, 1034) have respective properties and are different. As depicted in FIG. 10, the electric field 1034 is stronger than the electric field 1032. Accordingly, in this example, the portion of the fixation device 1010 exposed to the electric field 1034 will be more rigid as compared with the portion of the fixation device 1010 exposed to the less stronger electric field 1032.

The variance of the electric field across a particular fixation device may be varied in any suitable manner. In FIG. 10, the electric field (1032, 1034) is constant along an upper portion of the electric plates (1026, 1026′) and then makes a step change to be stronger along a lower portion of the electric plates (1026, 1026′). However, an applied electric field may be varied in any suitable manner. For example, the electric field may be progressively and gradually varied between sections of electric plates.

The different electric fields may be generated in any suitable manner such as by providing electric plates with varying thickness of the electric plates (as depicted in FIG. 10), by varying the application of electric current across the electric plates, or in some other suitable manner. Thus, the variance of an electric field may be varied by the properties of the plates (1026, 1026′) themselves or by the controller 1024.

In this embodiment, the controller 1024 is provided with a programmable portion 1025. The programmable portion 1025 is programmable such that a user may control and vary the strength of an electric field over time and/or vary the particular portions of the electric plate to which the electric field is applied.

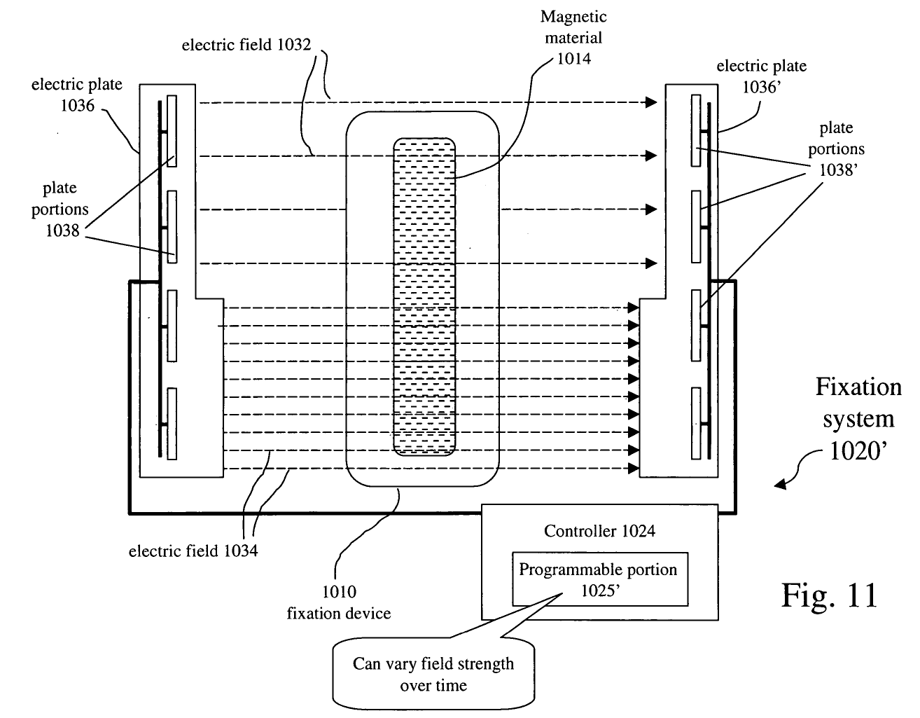

In further illustration of the above features, the fixation system 1020′ of FIG. 11 is of similar construction to the fixation system 1020 of FIG. 10. However, the fixation system 1020′ includes a modified programmable portion 1025′. The programmable portion 1025′ individually controls the application of respective electric fields across the area of plates (1036 and 1036′). That is, fixation system 1020′ is provided with electric plates (1036, 1036′). The electric plates (1036, 1036′) respectively include plate portions (1038, 1038′). In this embodiment, the programmable portion 1025′ individually controls each of the plate portions (1038, 1038′) so as to vary the electric field (generated by each of the electric plates) as desired. It is appreciated that known programming techniques may be used to implement the programmable portion 1025′ so as to control various attributes of the electric field or fields, i.e., such as varying the duration, timing, position, intensity, or other attribute of the electric field. In association therewith, known arrangements may be used to provide the electric plates used to generate the electric field or fields. Also, other known arrangements may be used to generate the electric field needed for application to the slurry.

In summary, in embodiments of the fixation device described above, the fixation device is subjected to an electric field. Once the electric field is turned on, the particles in the slurry align and as a result resist a change in position, i.e., resist deformation of the fixation device in which the slurry is housed. That is, without the electric field, the magnetic particles are in the slurry and are free floating. However, upon application of the electric field, the particles line up and become oriented with the electric field, and resist movement out of such alignment with the direction of the electric field. The stronger the electric field, the more resistant to forces the fixation device will become.

Further, as described above, a substantial degree of control is provided by the above arrangements. For example, a fixation device may be put in place and an electric field applied so as to make the fixation device very rigid. Then, as the bone calcifies, the rigidity of the fixation device may be decreased, i.e., by decreasing the electric field. As a result the bone will be progressively allowed to carry more of a normal load until the electric field is ultimately turned off, thus resulting in a healthy bone. The variance of the electric field may be programmed in upon initial placement of the fixation device, or alternatively, the electric field may be varied based on observed healing of the body component. In this manner embodiments of the fixation device provide great flexibility in the degree of support provided to a body component.

A variety of arrangements have been described above, as well as various features in such arrangements. It is appreciated that the various features in a particular embodiment may be combined with features in other embodiments as desired. Accordingly, for example, a fixation system having an electric field that varies over time may be combined with a fixation device having different magnetic particles in different portions of the fixation device.

As noted above, it is of course appreciated that various attributes of the fixation device 910 may be varied as desired, including the number of pockets, the composition of the magnetic particles and slurry in the pockets, the number of pockets that have different slurry, the shape or construction of the pockets and the relative orientation of the pockets, for example. It is also appreciated that features of the invention may be used to modify known fixation devices. For example, a particular component of a known fixation device may be replaced with a component comprised of an elastic structure, forming a pocket in which a slurry of magnetic particles is disposed. For example, a known nucleus replacement may be provided with a fixation device using the slurry as described herein, i.e., so as to control the rigidity of the nucleus replacement.

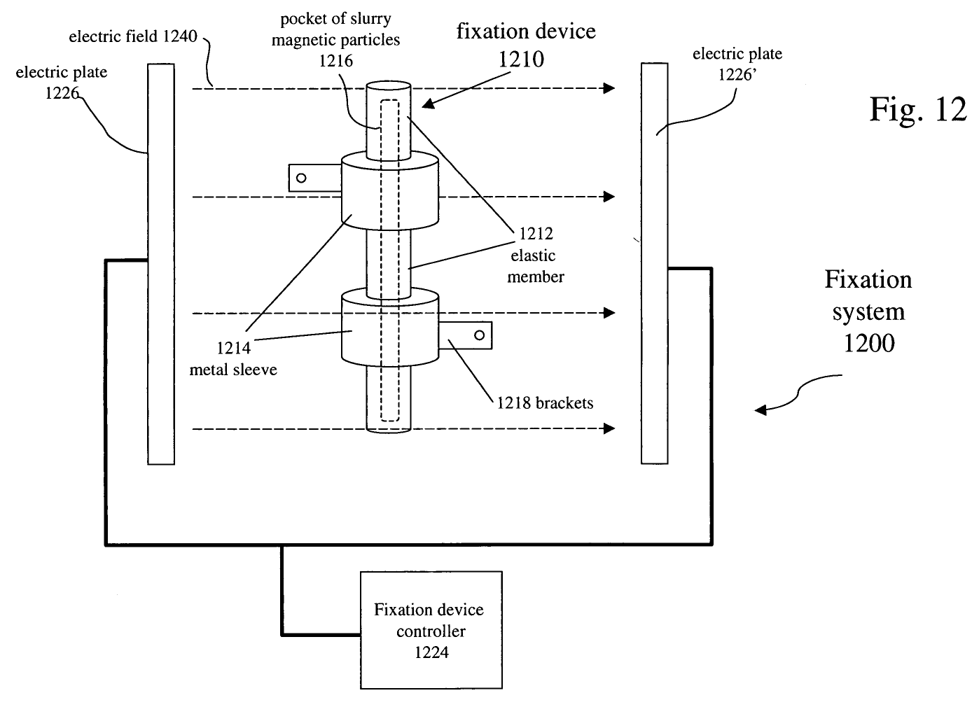

Further, a fixation device (provided with a pocket of slurry and of a desired shape) may be provided with suitable fastener arrangements as desired, such as grommets, screws, clamps or any other suitable fastener. In one embodiment as shown in FIG. 12, a fixation system 1200 includes a fixation device controller and electric plates (1226, 1226′) that apply and electric field 1240. A fixation device 1210 is disposed in the electric field.

The fixation device 1210 includes a rod shaped elastic member 1212. The elastic member 1212 contains a pocket of slurry 1216. The pocket of slurry 1216 includes magnetic particles as described above. The fixation device 1210 further includes a plurality of metal sleeves 1214. The metal sleeves 1214 may be cylindrical in form and encircle the elastic member 1212. Accordingly, the metal sleeves 1214 serve to support the elastic member 1212.

As shown in FIG. 12, the fixation device 1210 further includes brackets 1218 attached to the metal sleeve 1214. The brackets 1218 may be constructed of metal or other suitable material, and may be integrally formed with or attached to the metal sleeve 1214 in some other suitable manner. The brackets 1218 may be provided with apertures or other arrangement such that the brackets may be attached as desired.

In implantation, the fixation device 1210 is positioned in a desired location in a patient and attached using the brackets 1218. Thereafter, in use, the rigidity of the fixation device 1210 is controlled by the exposure of the fixation device to the electric field 1240, as described above. The shape of the fixation device 1210 may of course be varied as desired. For example, the fixation device 1210 may be provided with a hollow interior portion in which a body component may be disposed.

As noted herein, the fixation device may be in a wide variety of shapes depending on the particular application and the particular body component to be supported. It is not of course necessary that the entirety of a particular fixation device include the slurry of magnetic particles. That is, it may be that only a portion or a particular component of a fixation device be filled with the slurry, i.e., such that the stiffness of such part may be modulated.

It will be readily understood by those persons skilled in the art that the present invention is susceptible to broad utility and application. Many embodiments and adaptations of the present invention other than those herein described, as well as many variations, modifications and equivalent arrangements, will be apparent from or reasonably suggested by the present invention and foregoing description thereof, without departing from the substance or scope of the invention.

Accordingly, while the present invention has been described here in detail in relation to its exemplary embodiments, it is to be understood that this disclosure is only illustrative and exemplary of the present invention and is made to provide an enabling disclosure of the invention. Accordingly, the foregoing disclosure is not intended to be construed or to limit the present invention or otherwise to exclude any other such embodiments, adaptations, variations, modifications and equivalent arrangements.

What is claimed is:

1. A fixation device for implantation in a patient to provide support to a body component, the fixation device comprising:
an elastic outer construct forming a pocket;
a slurry disposed in the pocket, the slurry comprised of magnetic particles;
wherein the slurry is responsive to an electric field to modulate stiffness of the slurry.

2. The fixation device of claim 1, wherein the elastic outer construct is composed of rubber.

3. The fixation device of claim 1, wherein the fixation device includes a connection portion for connecting the fixation device to a body component.

4. The fixation device of claim 1I wherein the fixation device is in the form of a spinal rod.

5. A system for implementing a fixation device for implantation in a patient to provide support to a body component, the system comprising:
a fixation device including:
an elastic outer construct forming a pocket; and
a slurry disposed in the pocket, the slurry comprised of magnetic particles;
a control system including:
a controller; and
an electric field generator, the controller being operatively connected to the electric field generator so as to control an electric field emitted from the electric field generator;
wherein the electric field generator generates an electric field to which the slurry is reactive, the slurry becoming progressively stiffer as further electric field is applied.

6. The system of claim 5, wherein the controller controls the electric field generator so as to vary the intensity of the electric field, so as to increase stiffness of the slurry.

7. The system of claim 5, wherein the controller controls the electric field generator so as to vary the duration of the electric field, so as to increase stiffness of the slurry.

8. The system of claim 5, wherein the electric field generator is in the form of two opposed electric plates.

9. The system of claim 5, wherein the electric field generator is in the form of an internally disposed device that emits an electric field.

10. The system of claim 9, wherein the electric field generator is disposed in the patient, and adjacent to the fixation device.

11. The system of claim 10, wherein:
the implanted device further includes a device wireless interface portion; and
the controller includes a controller wireless interface portion; and
the device wireless interface portion interfaces with the controller wireless interface portion to control the application of the electric field from outside the patient.

12. A method for using a fixation device of modulated rigidity within a patient, so as to support a body component of the patient, the method comprising:
providing a fixation device having a compartment, the compartment filled with a slurry, the slurry comprised of magnetic particles;
positioning the fixation device in the patient adjacent the body component to be supported;
applying an electric field so as to expose the fixation device to the electric field, the slurry reactive to the electric field such that exposure of the slurry to the electric field orients the magnetic particles in the slurry to increase the rigidity of the fixation device.

13. The method of claim 12, wherein the compartment is formed by an elastic member.

14. The method of claim 12, wherein the electric field is applied by an electric field generator, the electric field generator being disposed external to the patient.

15. The method of claim 12, wherein the electric field is applied by an electric field generator, the electric field generator being disposed internally in the patient.

16. The method of claim 15, wherein the electric field generator is controlled by a controller that is disposed externally to the patient.

17. The method of claim 12, wherein the electric field is varied so as vary the rigidity of the fixation device over a period of time.

18. The method of claim 17, wherein the rigidity of the fixation device is varied over a period of weeks and in conjunction with healing of the body component.

19. The method of claim 12, wherein the compartment includes at least two sub-compartments, each sub-compartment being filled with the slurry.

20. The method of claim 19, wherein a first slurry in a first sub-compartment has different properties than a second slurry in a second sub-compartment, such that the first slurry and the second slurry provide different rigidity to respective portions of the fixation device.

21. The method of claim 19, wherein a first slurry in a first sub-compartment is exposed to a different electric field than a second slurry in a second sub-compartment, such that the first slurry and the second slurry provide different rigidity to respective portions of the fixation device.

22. The method of claim 21, wherein the different electric field is different based on the intensity of the respective electric fields.

23. The method of claim 12, wherein the magnetic particles includes a ferromagnetic material.

24. The method of claim 12, wherein the applying an electric field so as to expose the fixation device to the electric field is performed using a controller, the controller being programmable so as to vary the electric field based on anticipated healing of the body component.
